(19) United States
(12) Patent Application Publication
(10) Pub. No.: US 2017/0252297 A1
(43) Pub. Date: Sep. 7, 2017

(54) Standardized Subcutaneous Allergen Immunotherapy

(71) Applicant: Steven Ronder Yu, South Windsor, CT (US)

(72) Inventor: Steven Ronder Yu, South Windsor, CT (US)

(21) Appl. No.: 15/062,069

(22) Filed: Mar. 5, 2016

(51) Int. Cl. A61K 9/00; G06F 19/00; A61K 39/35

(52) U.S. Cl. CPC A61K 9/0019; A61K 39/35; G06F 19/326

(57) ABSTRACT

The invention provides a process for the standardization of subcutaneous allergen immunotherapy formulas resulting in increasing availability and decreasing costs, allowing for expanded and improved relief for a greater number of allergy sufferers.

This invention provides a lower cost treatment to increase availability of the benefits of subcutaneous immunotherapy via standardization of most common seasonal or year round allergens for treatment. Standardizing a formula containing multiple allergen types allows a large population of potential users to benefit from a wide spectrum of included allergen sensitivities. Mass production of non-customized formulas allows for cost reduction, which breaks down a major barrier to current demand. The availability of non-individualized reduced allergen extract concentration strength formula allows for cost-effective availability in less-specialized delivery channels, which breaks down the other major barrier to current demand.

### BACKGROUND OF THE INVENTION

Subcutaneous allergen immunotherapy has long been known and used. The treatment results in highly preferred outcomes when compared to over the counter allergy treatments such as antihistamine or corticosteroid medications. Compared to antihistamine or corticosteroid treatments, allergen immunotherapy is considered to be more effective and with fewer unwanted side effects.

However, typical subcutaneous treatment involves highly customized treatment formulas of allergen extracts individually specified to the patient and with formulas also dependent on phase of treatment, i.e. build-up phase and maintenance phase. Each phase requires a different customization of the specific allergen extract concentration. The highly customized allergen treatments for individuals requires allergen immunotherapy experts to establish standard practices to minimize liabilities.

In addition, due to the risk of anaphylactic shock, each patient must remain in the observation of the medical professionals immediately after the dose is administered for a significant period of time. Therefore, current treatment methods require a significant output of time in order to receive the immunotherapy, including the actual procedure, the waiting time, and any additional travel time to the location. The highly customized allergen treatments for individuals requires allergen immunotherapy experts to establish standard practices within the medical offices to minimize liability.

The high cost of the formula, combined with the time requirement to receive the subcutaneous allergen immunotherapy significantly reduces the demand for the otherwise highly preferable treatment. As a result, the number of medical offices specializing in the practice are few, and therefore the far distance to be traveled increases the time commitment necessary to receive the treatment, again resulting in reduced demand.

Finally, with the advent of high deductible health plans, the cost for elective immunotherapy treatment has become significantly more visible to those under the high deductible health plans to add more pressure to seek lower cost forms of relief via over the counter medication.

All of these factors lead to the current environment where many of the patients seeking subcutaneous allergen immunotherapy are suffering with debilitating or life-threatening allergies, while most of remaining population of allergy sufferers are utilizing less preferable forms of treatment.

### SUMMARY OF THE INVENTION

The most common year-round allergens would be identified via the Pareto rule for selection to make up a standard formula for subcutaneous delivery. This formula with standard extracts makeup would be able to be mass-produced. Separately, a low strength concentration of this standard formula would be mass-produced as a standard single dose vaccine. This low concentration formula could be offered as a lower cost non-customizable option for patients suffering from a range of allergens. This standard formula could be administered in medical offices or pharmacies with medical professionals qualified to administer a vaccine such as the influenza vaccine, but who would otherwise not have the adequate safety infrastructure to administer customized formulas. Similarly, a seasonal vaccine formula for spring and fall allergy seasons could be developed without overlapping with the standard year-round formula,

### DETAILED DESCRIPTION OF THE INVENTION

Using historical data, the initial standard year-round formula could be developed to provide maximum coverage of patients. Depending on the cost, the number of allergens to be included in the standard formula may be reduced if there is a substantial savings to do so, as the reduction of any allergen would reduce the potential base of demand. However, the cost of an individual extract may not be the primary decider for inclusion into the standard formula, as other considerations such as risk and efficacy should be determined.

### EXAMPLE 1

### The Common Year-round Allergens: Pollen from Different Plants, Dust Mites or Other Household Pests, Mold, Animal Dander, Insect Stings, Latex, and Certain Food and Medications, Although Currently Food Allergies are Not Considered as Treatable via Subcutaneous Allergen Immunotherapy

### EXAMPLE 2

### The Common Seasonal Allergens: Pollen from Different Plants and Different Molds

Upon finalization of the standardized year-round and seasonal formulas, the standardized dosing/concentration strength must be determined. The dosing/concentration strength should be calculated as to provide the most patients with adequate relief for a determined period of time, such as a minimum of a few days up to a week. As long as availability of delivery channels can be significantly increased and the cost per dose significantly reduced, an increased dose frequency may not be a significant obstacle when compared to the imposition of the build-up and maintenance phase procedure that accompanies the customized formula method.

Note 1: the initial release may be either for a seasonal or year-round formula, or possibly both. Whichever release approach is determined as having the best outlook for reaching adequate distribution would create a greater market for subsequent releases of other non-overlapping formulas.

Note 2: the initial standardized formulas determined via analysis will contain many of the common allergens and the specific makeup of the formula is therefore likely to already be in existence as a customized formula for certain individuals currently or in the past. As clarification, this invention is in the process by which the standard formula is determined to be beneficial to the largest population of allergy sufferers and is thereupon mass produced for wide distribution at a much lower cost than the current formulas that are prepared for individual customization.

Note 3: the current subcutaneous allergen immunotherapy regimen consists of the starting phase where low-concentration formulas of allergen extracts are used prior to building up to higher concentration formulas of allergen extracts. The low-concentration formulas are safer but provide less relief, while the high-concentration formulas provide maximum relief in terms of symptoms and duration. However, it is common for the low-concentration formula to provide significantly better relief compared to treatment via over the counter antihistamines. The invention is applicable to the current state where allergen overreaction is to be avoided for safety, while aiming to provide adequate relief for a few days to the greatest number of allergy sufferers.

Note 4: the invention considers that standardized higher-concentration formulas of allergen extracts may be released as demand and economics allow for this development. The standardized higher-concentration formulas would provide improved relief relative to the lower concentration formula while remaining cost effective and highly available, as long as the medical office administering the formula has adequate safeguards for managing the greater risk. These safeguards exist within the current delivery channels such as Ear Nose Throat specialty medical offices.

Note 5: the expectation would be for the initial standard formula to be revised after more data is gathered regarding the larger population of allergy sufferers. As the current percentage of sufferers undergoing detailed allergen testing may be much smaller than the total population, the standardization would change as data of the other group begins to emerge. In addition, as allergies have increased in recent years, there is no certainty that the current profile of allergens will not change over time.

1. The establishment of standardized subcutaneous formulas
Using existing data and gathering data to determine the currently known spectrum of allergen sensitization
Using existing data and gathering data to determine the optimal low concentration of extracts to provide adequate relief (more than a few days) while minimizing risk of significant reactions
Using the two above data points to define an initial standard formulas and concentration strength for mass-production (1 or more depending on the data)
Improving the formulas to be more effective and to reach a larger population of allergy sufferers, in some cases by increasing the number of standard formulas or concentration beyond one (1) formula and concentration level, where demand and delivery channels can support the expansion

2. Mass production of a standard delivery mechanism of the subcutaneous allergen immunotherapy formula and concentration

3. Submitting the standard subcutaneous allergen immunotherapy formulas and concentrations into the marketplace for administration by medical professionals
Where and when possible, expanding the delivery channels beyond the current Ear Nose and Throat specialty medical offices
The availability of vaccines at pharmacies is noted as a model for the increase in delivery channels available for administering the standard formulas in a controlled environment where the patient may be observed for the adequate period of time
The standardized formula could also be available at workplace on-site medical offices where they could be administered during break periods
